(19) United States
(12) Patent Application Publication
(10) Pub. No.: US 2024/0296940 A1
(43) Pub. Date: Sep. 5, 2024

(54) UNIQUE HCP USER IDENTITY SYSTEM

(71) Applicant: HARSHIT JAIN, PARSIPPANY, NJ (US)

(72) Inventor: HARSHIT JAIN, PARSIPPANY, NJ (US)

(21) Appl. No.: 18/436,013

(22) Filed: Feb. 7, 2024

Related U.S. Application Data
(63) Continuation-in-part of application No. 18/395,123, filed on Dec. 22, 2023.
Application No. 18/395,123 is a continuation-in-part of application No. 18/116,290, filed on Mar. 1, 2023, now Pat. No. 11,869,673.
(63) Continuation-in-part of application No. 18/379,056, filed on Oct. 11, 2023.
(63) Continuation-in-part of application No. 18/078,953, filed on Dec. 10, 2022.
(63) Continuation-in-part of application No. 17/857,013, filed on Jul. 3, 2022.

(51) Int. Cl. G16H 40/20; G16H 10/00

(52) U.S. Cl. CPC G16H 40/20; G16H 10/00

(57) ABSTRACT

The embodiments disclose a unique healthcare professional user identity system including an identity solution database configured for collecting end user healthcare professional (HCP) digital device actions signals, an identity resolution server coupled to the identity solution database configured to collate the client side end user HCP digital device actions signals, an analyzer coupled to the identity resolution server configured for performing data targets/analytics of the HCP digital device actions signals, a processor coupled to the identity resolution server configured to identify behavioral patterns based on browser attributes of the HCP digital device actions signals, wherein the at least one processor generates and assigns a unique user identity (UUID) to the identified HCP, the HCP identified behavioral patterns, and existing and future HCP consent forms, and wherein the identity resolution server is configured to provide the behavioral patterns data to advertiser partners for the newly determined advertising targeting related data.

### CROSS-REFERENCE TO RELATED APPLICATIONS

This Patent Application is a Continuation-in-part and claims priority to the United States Patent Application entitled: “HCP CONSENT MANAGEMENT FRAMEWORK 200 SYSTEM”, U.S. Ser. No. 18/395,123 filed Dec. 22, 2023, by Harshit Jain, which is a Continuation-in-part of United States Patent Application entitled: “ELECTRONIC HEALTH RECORD PLATFORM”, U.S. Ser. No. 18/116,290 filed Mar. 1, 2023, by Harshit Jain, all of which are incorporated herein by reference.

### BACKGROUND

Understanding and traversing the user identity landscape is a cumbersome and complex process. The complexity increases multifold whenever there is a need to resolve the identity of an online user who is also a Healthcare Professional (HCP). The parameters for identifying an HCP and their online activities across various digital assets such as websites, Electronic Health Record systems (EHRs), healthcare professional-specific digital libraries and medical reference content, and Point-of-care (POC) applications, are indeed complex. Moreover, an HCP might be, in their specific line of duty, switching frequently between devices and healthcare applications. In such a case the idea of identifying a particular HCP across their digital footprint over a given period of time, say, even a single day becomes a complicated proposition to accomplish and maintain within a specific digital system.

Identifying the HCP (healthcare professional) identity landscape across geographies is a big challenge for publishers to overcome. Commercial and advertorial messaging is a highly niche segment that is strictly governed by prescribed rules, compliances, and regulations. The majority of these restrictions have been enacted by medical associations and healthcare regulatory bodies in particular nations and markets. Furthermore, the message content is governed by stringent laws. Additionally, there are stringent rules that prohibit the display of certain drugs and medical formulations to the general populace. Similarly, there are other fraudulent and non-medical compositions whose messaging is prohibited by legislation all over the world. Further, the healthcare professional has limited time to search for commercial and advertorial messaging that is relevant to their specific line of duty. Identifying individual healthcare professionals usually includes their area of specialization to appropriately target the medical product content information while complying with geographical compliance requirements.

### BRIEF SUMMARY OF THE INVENTION

The unique HCP user identity system is used for identifying HCPs based on their behavior footprint. HCP behavior determinations need to be collected. To begin with, it is important to know whether the user is an HCP or not. The parameters for identifying that the user is associated with the healthcare or life sciences domain forms the cornerstone of the unique user identity generation logic. Once it is established that the user is an HCP then various other professional aspects around the HCP need to be investigated and established before this information can be put to viable commercial use and for the benefit of all the parties concerned.

After establishing the fact that an individual is an HCP, the next step is identifying what area or field of medicine these HCPs perform. Determine with what branch of medical science the HCPs are associated with, and what specialization, if any. Discover any specific training in their area and has the HCP established themselves as an expert in that field of medical science.

Analyze the HCP behavioral pattern concerning their profession. Behavior patterns of the time of the day they are working; are their work hours fixed or are they specialists and operate on a need basis. Are there HCP face-to-face interactions with patients; what type of ailments and diseases do they treat; and what prescription methodology or workflow the HCP follows during caregiving sessions at their specific workplace. HCPs spend time constantly looking forward to updating and upgrading their current knowledge. Sources where the HCP is seeking new information about innovations in their definite area of specialization.

### BRIEF DESCRIPTION OF THE DRAWINGS

FIG. 1 shows for illustrative purposes only an example of the publisher website consent management of one embodiment.

FIG. 2 shows for illustrative purposes only an example of the HCP Consent Management Framework of one embodiment.

FIG. 3 shows for illustrative purposes only an example of a consent collection form of one embodiment.

FIG. 4 shows for illustrative purposes only an example of a bespoke user experience of one embodiment.

FIG. 5 shows for illustrative purposes only an example of an opt-out review of one embodiment.

FIG. 6 shows for illustrative purposes only an example of access and transparency of one embodiment.

FIG. 7 shows for illustrative purposes only an example of compliance of one embodiment.

FIG. 8 shows for illustrative purposes only an example of trust building of one embodiment.

FIG. 9 shows a block diagram of an overview of third-party relationships of one embodiment.

FIG. 10 shows for illustrative purposes only an example of consent synchronization of one embodiment.

FIG. 11 shows a block diagram of an overview of the consent matrix of one embodiment.

FIG. 12 shows for illustrative purposes only an example of the cookie consent/tracking mechanism of one embodiment.

FIG. 13 shows for illustrative purposes only an example of a unique HCP user identity of one embodiment.

FIG. 14 shows for illustrative purposes only an example of client-side user identifiers of one embodiment.

FIG. 15 shows for illustrative purposes only an example of one HCP user and one UUID of one embodiment.

FIG. 16 shows for illustrative purposes only an example of probabilistic and deterministic identifiable signals of one embodiment.

FIG. 17 shows for illustrative purposes only an example of identity enrichment of one embodiment.

FIG. 18 shows for illustrative purposes only an example of advertising targeting of one embodiment.

FIG. 19 shows for illustrative purposes only an example of cross-device tracking of one embodiment.

FIG. 20 shows for illustrative purposes only an example of personalization (user experience) of one embodiment.

FIG. 21 shows for illustrative purposes only an example of data monetization of one embodiment.

FIG. 22 shows a block diagram of an overview of other benefits of one embodiment.

### DETAILED DESCRIPTION OF THE INVENTION

In a following description, reference is made to the accompanying drawings, which form a part hereof, and in which is shown by way of illustration a specific example in which the invention may be practiced. It is to be understood that other embodiments may be utilized and structural changes may be made without departing from the scope of the present invention.

It should be noted that the descriptions that follow, for example, in terms of an HCP Consent Management Framework system are described for illustrative purposes and the underlying system can apply to any number and multiple types of website providers. In one embodiment of the present invention, the HCP Consent Management Framework system can be configured using consent codes. The HCP Consent Management Framework system can be configured to include overall consent forms and can be configured to include a granular consent form using the present invention.

Message utilization poses another significant challenge for publishers. Matching and aligning the appropriate commercial message with the right HCP is a complex and time-consuming endeavor. For instance, extensive research is conducted to identify the HCPs, ensuring that an advertisement with cardiology-related medical product content information reaches an HCP specialized in cardiology rather than one specialized in a different medical field, such as dentistry or orthopedics. In the future, there will be a growing need for healthcare and life sciences marketers to access information about recent and innovative medical solutions and effectively reach the corresponding HCPs and medical practitioners on a large scale.

Publishers are also under considerable pressure to conceive, design, and establish their own customized HCP Consent Management Frameworks. This is essential for expanding HCP identities and, in turn, driving more targeted traffic to their digital advertising spaces.

Additionally, a significant volume of medical product content information, authored by experts in the medical field, is intended solely for consumption by specialist medical practitioners. In such cases, a consent management mechanism plays a pivotal role in restricting public access to this medical product content information. Moreover, publishers may limit access to detailed medical product content information on their websites exclusively to pre-identified HCPs, often tailoring the experience for HCPs who visit specific sections or pages.

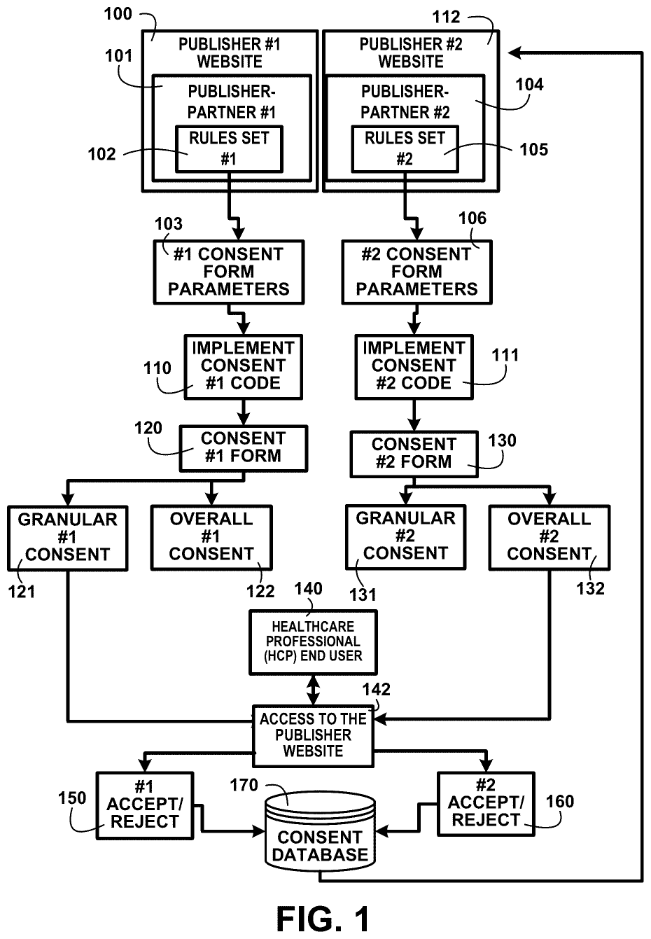

FIG. 1 shows for illustrative purposes only an example of a publisher website consent management of one embodiment. FIG. 1 shows a publisher #1 website 100 having a publisher-partner #1 101 selecting rules set #1 102 and #1 consent form parameters 103. The publisher #2 website 112 has a publisher-partner #2 104 selecting rules set #2 105 with #2 consent form parameters 106. Each publisher-partner provides consent form parameters based on their website medical product content information and geographical compliance requirements. In one embodiment, the #1 consent form parameters 103 are converted into code to implement consent #1 code 110. The consent code is converted in at least one consent [symbol] #1 form 120. For example, consent [symbol] #1 form 120 is converted into an overall #1 consent 122 to cover all medical product content information of the publisher-partner #1 101. In addition, consent [symbol] #1 form 120 is converted into multiple granular #1 consent 121 forms. Each granular #1 consent 121 form covers specific aspects of the publisher-partner #1 101 medical product content information.

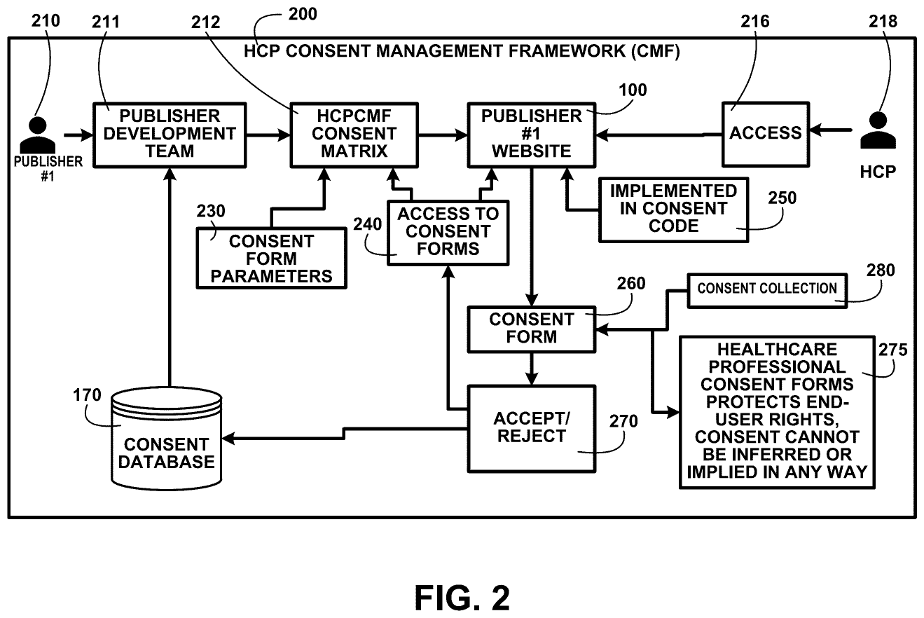

As discussed in U.S. application Ser. No. 18/116,290, which is incorporated by reference herein, the medical products graphical user interface is configured to display medical product content information on electronic health records medical provider and patient interaction segment screens during an appointment. A healthcare professional (HCP) end user 140 may only consent to medical product content information or another offering listed on the granular #1 consent 121 form. The healthcare professional (HCP) end user 140 will receive the consent forms upon access to the publisher's website 142. The HCP 218 of FIG. 2 may #1 accept/reject 150 one or more consent forms. The election by the HCP 218 of FIG. 2 is recorded on the consent database 170 of the publisher #1 website 100. In this example, the HCP 218 of FIG. 2 has elected one of the granular #1 consent 121 form selections.

In another embodiment, the #2 consent form parameters 106 are converted into implement consent #2 code 111. At least one consent [symbol] #2 form 130 into a granular #2 consent 131 and an overall #2 consent 132 form. The HCP upon access to the publisher website 142 selects the overall #2 consent 132 form and responds to the #2 accept/reject 160 accept. The accepted overall #2 consent 132 form is recorded in the consent database 170 of the publisher #1 website 100 of one embodiment. Message utilization is another great challenge that publishers need to address on their end. It is a massive and time-consuming endeavor for publishers to just match and map the suitable commercial message with the correct HCP 218 of FIG. 2. For example, much research goes into HCP 218 of FIG. 2 identification so that an advertisement with a context related to cardiology reaches an HCP 218 of FIG. 2 who specializes in cardiology rather than reaching an HCP 218 of FIG. 2 who specializes in another medical specialty, for example, dentistry or orthopedics.

There is a requirement for marketers in the healthcare and life sciences domain to get information about recent and innovative medical solutions to reach the corresponding HCPs and medical practitioners at the earliest and with the best possible means to reach HCPs and that too at scale.

There is a high demand on the publisher 210 of FIG. 2 side to conceptualize, design, and develop their bespoke HCP 218 of FIG. 2 Consent Management Frameworks to scale HCP 218 of FIG. 2 identities and thereby drive more targeted traffic to their respective digital advertisement spaces.

Moreover, there is a growing body of medical product content information that is created by experts in the medical field and is meant to be consumed by none other than specialist medical practitioners. In such a scenario, a consent management mechanism plays a significant role in preventing the general public from accessing such medical product content information. Furthermore, publishers may restrict access to granular medical product content information on their websites only to previously identified HCPs, to the point of generating custom-made experiences for HCPs landing on particular sections or pages.

FIG. 2 shows for illustrative purposes only an example of the HCP Consent Management Framework (CMF) 200 of one embodiment. FIG. 2 shows an HCP Consent Management Framework 200 to collect consent from HCP 218 users of a publisher #1 website 100. The Publisher 210 has a publisher development [symbol] team 211 to establish an HCPCMF consent matrix 212 on the Publisher #1 [symbol] website 100. Access 216 from an HCP 218 is authenticated referencing consents granted by the HCP 218 stored in the consent [symbol] database 170. No consent recorded, triggers consent form parameters 230 implemented in consent code 250 in the form of access to consent forms 240 converted into one or more consent [symbol] forms 260. Consent forms refer to medical product content information that can be accessed by the HCP based on the explicit consent they have provided to the Publisher on a specific website. The HCP 218 user may accept/reject 270 from a selection of consent forms and add the HCP 218 selection into the consent collection 280. Healthcare professional consent forms protect end-user rights, consent cannot be inferred or implied in any way 275 of one embodiment.

The HCP Consent Management Framework 200 promotes the concept of consent granularity, ensuring that healthcare professionals (HCPs) have the autonomy to select the specific types of data processing and online marketers with whom they are willing to share their data. This level of granularity empowers HCPs to exercise greater control over the data they provide to publishers. Once consent is obtained, the data is processed by the HCPs' agreed-upon terms.

HCPs may be required to provide consent for various activities, which can vary among different publishers based on their unique business needs. The list of activities for which HCP 218 consent is required is presented to the HCP 218 in a digital format, often as a pop-up window when they visit the publisher's website. The consent form is designed to be clear and straightforward, ensuring that HCPs can easily understand and provide their consent.

The form would mention the activities for which explicit consent is being sought and the language used in the form would be straightforward for the HCP 218 to understand.

The HCP Permission Management Framework provides the publishers with a template for developing and specifying the portions of the consent form they wish to display to the HCPs. The form design template also allows the publishers to include their consent statements. The template will also enable publishers to develop a consent form that is not only concise but also simple enough for HCPs to easily understand and grant their approval accordingly.

Activities for which consent is being sought are presented as a list of items with a corresponding checkbox for each of the listed activities that the HCP 218 can tick/select. The goal is to offer the information to the HCP 218 in such a way that they can freely select required statements or settings expressing their permission for the selected information to be processed and used by the publisher 210 to conduct the indicated activities.

The HCP Permission Management Framework's design and development both promote end-user rights because the consent form does not provide pre-selected options to the user or a mechanism by which publishers can create a form in which inactivity of the HCP 218 is in any way considered consent. In doing so, the publisher 210 never violates any authorized data processing provisions of regulatory frameworks, which solely considers willfully granted consent.

Additionally, publishers may easily show this consent form, as valid documentary proof or evidence, to the data regulatory authorities of their various regions that the consent was obtained voluntarily and without any compulsion or using any unfair means, from the concerned HCPs.

Based on the content that is taken from HCPs, the corresponding publishers would then apply the consent to the entire website (site-level consent ramifications); or the consent would be applied at the web page level (HCPs can be provided or denied access to certain pages of the website based on the received HCP 218 content), or the consent could be enforced for certain sections of the website's page (for example, a section dedicated to a content snippet meant for cutting edge research in oncology would not be exposed to an HCP 218 that is a specialist in dermatology or say nephrology) based on the attributes shared by the concerned HCP 218.

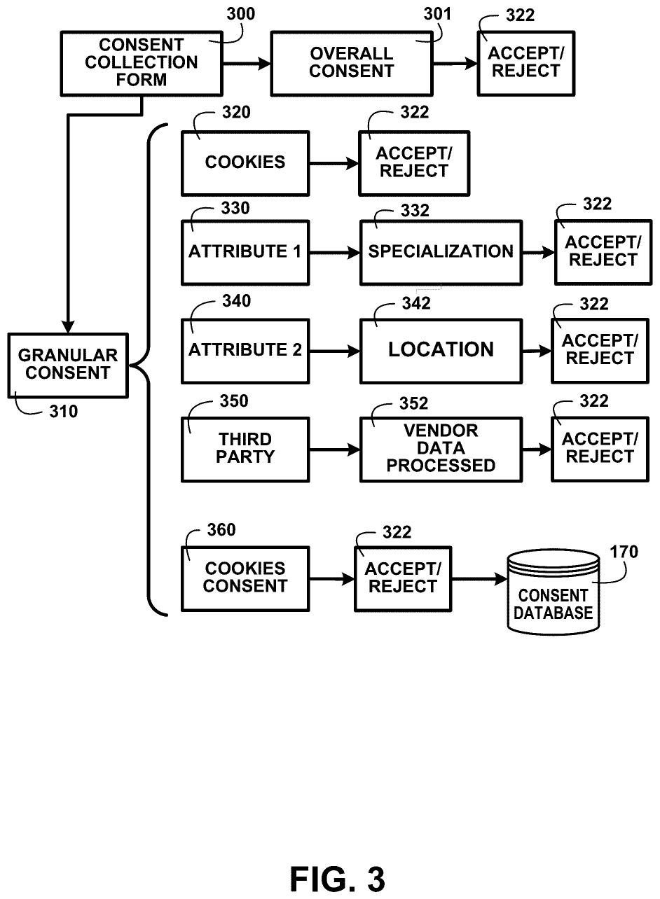

FIG. 3 shows for illustrative purposes only an example of a consent collection form of one embodiment. FIG. 3 shows an example of a consent collection form 300 content. In one example, an overall consent 301 the HCP 218 of FIG. 2 user may accept/reject 322. Another example is a selection of granular consent 310 forms. In one embodiment, the granular consent 310 form may include cookies 320 that display using a graphical user interface (GUI) to list what medical product content information the cookie form includes that the HCP 218 of FIG. 2 user may accept or reject 322.

Other examples of a granular consent 310 form include attribute 1 330 of the HCP 218 of FIG. 2 user being specialization 332 to accept/reject 322. Another attribute 2 340 may include a geographical location 342 to accept/reject 322. Medical product content information consent varies by geographical location and the granular consent 310 form will include specific content consents based on geographical compliance regulations. Another issue is third-party 350 content and the manner the vendor data processed 352 for display. The HCP 218 of FIG. 2 may accept/reject 322 based on location compliance restrictions. Cookies consent 320 is subject to acceptance based on the medical product content information of the cookie displayed to select accept/reject 322 responses. Any of the consent responses received is stored in the consent [symbol] database 170 of one embodiment.

The HCP Consent Management Framework 200 of FIG. 2 would store/document consent details of the HCPs for the respective publisher 210 of FIG. 2 within a consent database. The consent collected from the HCPs would also be accompanied by the corresponding attributes they share, for example, specialization, location, etc. Moreover, the attributes collected from the HCPs are configurable, which means that the HCPs can freely select the specific consent they want to provide and would subsequently be used for the stated data processing and for receiving messages and medical product content information accordingly.

The HCPs would be asked to provide specific attributes around their nature of work, that is, with which medical specialty they are aligned. They could also be asked to provide information about their location or specifically the location of their workplace, that is, where the hospital, clinic, or healthcare facility they are associated with is located. The HCP 218 of FIG. 2 could also be asked to provide consent for any specific academic objective that they would like to pursue or any specific publication they would like to subscribe to.

These attributes would vary from one publisher 210 of FIG. 2 to another and even differ from one country to another within a specific geographically demarcated market. Nonetheless, the publishers would only seek the information from the HCPs that is legal within their specific market/country/location. In doing so, publishers do not infringe on any compliance parameters and adhere to the prescribed medical and legal injunctions of their respective regions or countries.

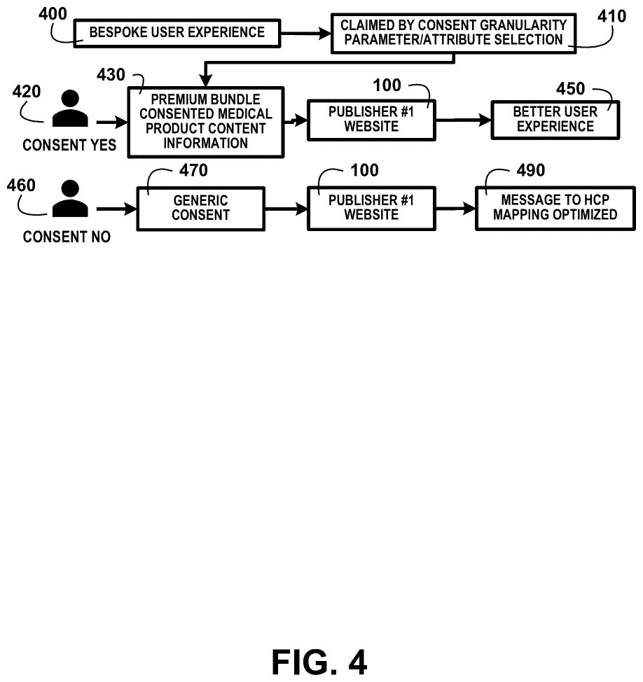

FIG. 4 shows for illustrative purposes only an example of a bespoke user experience of one embodiment. FIG. 4 shows bespoke user experience 400 claimed by consent granularity parameter/attribute selection 410. An HCP 218 of FIG. 2 consent yes 420 response to a premium bundle of consented medical product content information 430 for the publisher #1 website 100 creates a better user experience 450 by consenting to all publisher medical product content information. An HCP 218 of FIG. 2 consent no 460 response to a generic consent 470 publisher #1 website 100 generates a message to HCP 218 of FIG. 2 mapping optimized 490 to avoid consent-required medical product content information of one embodiment.

Based on the consent provided by consenting to all publisher medical product content information or only HCP-selected portions of the publisher medical product content information, an HCP can be shown or debarred from accessing medical product content information on a specific website. This means HCPs excelling in a particular healthcare domain get to see medical product content information associated with their specific areas of expertise only and not just any other generic medical product content information. For HCPs who do not provide explicit consent, the website through providing them access to the generic medical product content information would restrict such users from accessing specific premium medical product content information present and available on the site. This means the consent provided by the HCP 218 of FIG. 2 would determine what type of medical product content information or message they would get to see on the publisher's website.

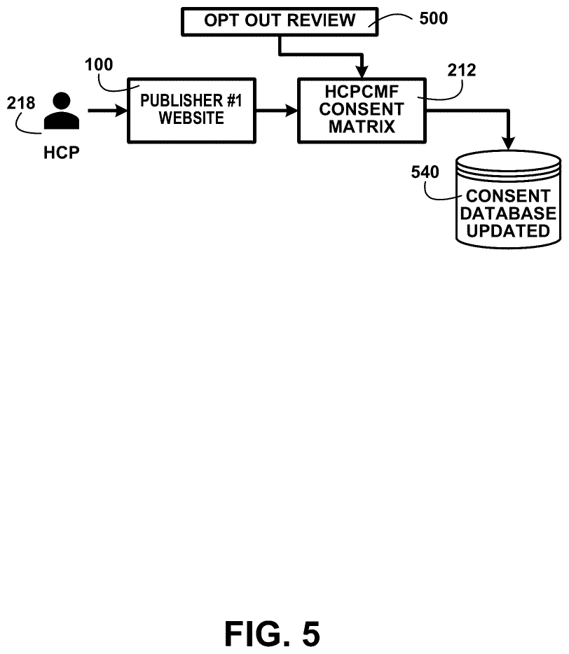

FIG. 5 shows for illustrative purposes only an example of an opt-out review of one embodiment. FIG. 5 shows an opt-out review 500 of an HCP 218 publisher #1 website 100 selection. The HCPCMF [symbol] consent matrix 212 records the opt-out response with a consent [symbol] database [symbol] updated 540.

Opt-Out Management: The HCP Consent Management Framework 200 of FIG. 2 allows HCPs to withdraw their consent whenever they see fit, respecting the rights of data subjects, i.e., the HCPs. The option to opt-out is a fundamental aspect of data subject rights and is a standard practice mandated by privacy regulatory authorities worldwide of one embodiment.

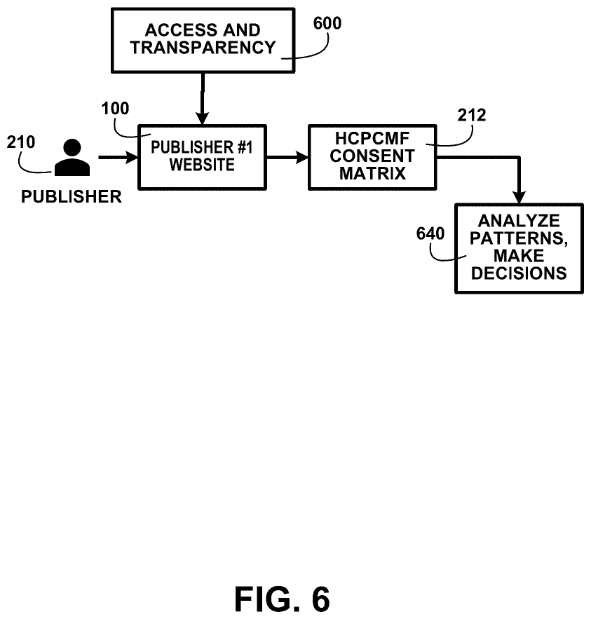

FIG. 6 shows for illustrative purposes only an example of access and transparency of one embodiment. FIG. 6 shows access and transparency 600 from a publisher 210 on the publisher #1 website 100. Further, as discussed in U.S. application Ser. No. 18/116,290, incorporated above, a medical products provider computer stored the medical product content information and graphical displays on network platform databases and memory devices coupled to network platform servers, communication devices, processors, graphic devices, and other digital devices. The HCPCMF [symbol] consent matrix 212 uses a platform having a plurality of servers and processors to analyze patterns and make decisions 640 related to HCP 218 of FIG. 2 user privacy and consent matters of one embodiment. HCPs are asked to provide specific attributes related to their professional background, including their medical specialty, workplace location, academic objectives, or publication preferences. The specific attributes may vary between publishers and even from one country to another within a geographically segmented market. Importantly, publishers only request information that is legally permissible in their specific market or country, ensuring compliance with medical and legal regulations.

The HCP Consent Management Framework 200 of FIG. 2 offers easy access for publishers, providing them with a dashboard to review their HCP 218 of FIG. 2 consent-related data. This facilitates a deeper understanding of consent data, enabling publishers to derive valuable insights and make informed business decisions. The HCP Consent Management Framework 200 of FIG. 2 is easy to access, and publishers can seamlessly review their respective HCP 218 of FIG. 2 consent-related data. For a better understanding of the consent data, to derive appropriate business acumen from consent data, and to make informed business decisions, publishers are provided with a dashboard.

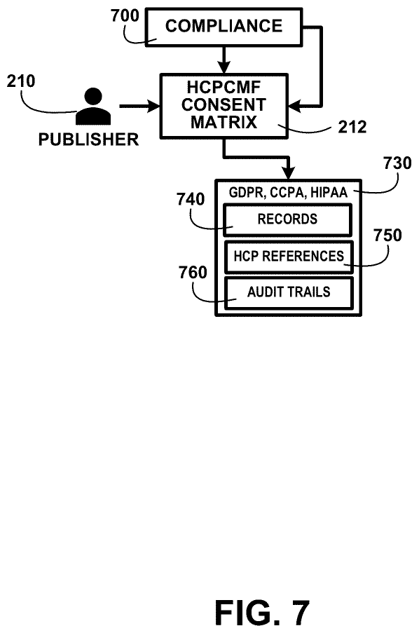

FIG. 7 shows for illustrative purposes only an example of compliance of one embodiment. FIG. 7 shows compliance 700 by the publisher 210 using the HCPCMF consent matrix 212 to meet regulatory compliance with at least GDPR, CCPA, and HIPAA 730. Where in the compliance agencies include at least GDPR (General Data Protection Regulation), CCPA (California Consumer Privacy Act) and HIPAA (Health Insurance Portability and Accountability Act) are some of the popular and cited regulatory frameworks, that govern data privacy and data protection procedures and process in the EU (European Union) region and the US, respectively.

The consent database 170 of FIG. 1 includes consent form records 740, HCP references 750, and audit trails 760 to verify to any compliance agency that the publisher 210 and publisher #1 website 100 of FIG. 1 are in compliance of one embodiment.

AdTech organizations, for example, advertisers or publishers operate in a complex legal framework, with national laws, medical laws, and restrictions imposing strict data processing obligations. The HCP Consent Management Framework 200 of FIG. 2 aids publishers in remaining compliant with these rules and regulations at all times. The compliance needs are met within the framework by transparently maintaining HCP 218 of FIG. 2 consent records, managing consent preferences, and giving audit trails to publishers. All of this enables publishers to give verifiable evidence of compliance to data compliance authorities in their specific geography.

Based on the content taken from HCPs, publishers can apply consent at different levels. This includes applying consent at the site level, web page level, or for specific sections of a webpage, depending on the attributes shared by the HCPs.

The HCP Consent Management Framework 200 of FIG. 2 ensures bespoke user experiences. HCPs are shown medical product content information related to their specific areas of expertise based on their consent. Those who do not provide explicit consent can access generic medical product content information but are restricted from accessing premium medical product content information.

The framework also prioritizes managing opt-outs, allowing HCPs to withdraw their consent as needed. This is vital for respecting the rights of data subjects, and the HCPs, and aligns with privacy regulations globally.

Respecting user rights is a core principle of the HCP Consent Management Framework 200 of FIG. 2. HCPs can change their consent preferences or withdraw consent without any hindrance. When an HCP 218 of FIG. 2 opts out or withdraws consent, the publisher 210 of FIG. 2 ceases to process their data, ensuring full compliance with data subject rights and privacy regulations.

The HCP Consent Management Framework 200 of FIG. 2 has been designed such that the rights of data subjects, that is, HCPs, are upheld both by design and by development. HCPs can withdraw their consent and even change their consent preferences as and when needed, and they can do so without any prior obligation or hindrance. Once an HCP 218 of FIG. 2 has opted out or withdrawn their consent from a website, the concerned publisher 210 of FIG. 2 stops processing the data of that particular HCP 218 of FIG. 2.

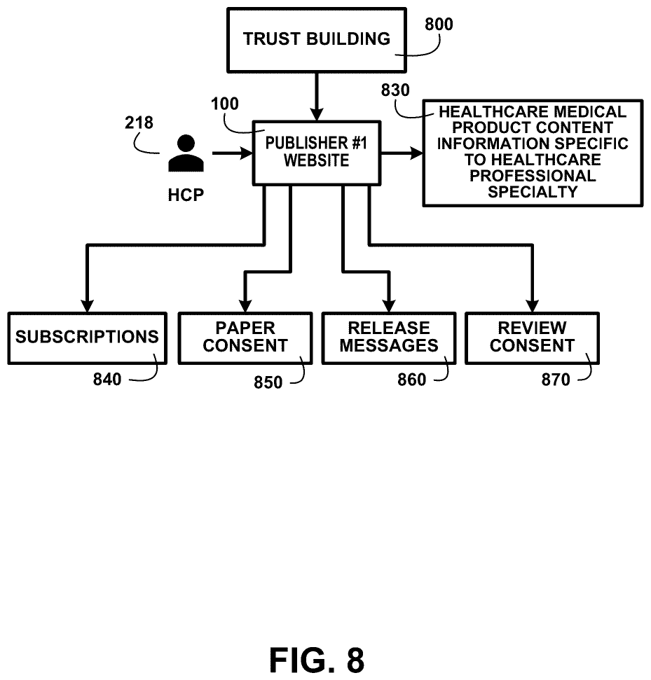

FIG. 8 shows for illustrative purposes only an example of trust building of one embodiment. FIG. 8 shows trust building 800 between the HCP 218 and publisher #1 [symbol] website 100 that provides healthcare medical product content information specific to healthcare professional specialty 830. The HCP 218 subscriptions 840 allow for paper consent 850 for relevant advertisements and medical product content information, information release messages 860 including commercial messages, and review consent 870 of medical publications and research papers by the HCP 218 of one embodiment.

The HCP Consent Management Framework 200 of FIG. 2 enables publishers to create trust with the HCPs they serve through their websites. This is accomplished through HCP 218-focused consent. This means that end users can only access healthcare-related material after providing explicit agreement that they are in the healthcare or life sciences domain.

Furthermore, by using consent granularity, publishers can offer HCPs messages that are specifically tailored to their needs. When HCPs are fully aware that they control their consent preferences and how their consent data is used by publishers who are providing them with relevant advertisements, commercial messages, and content, they are more likely to interact with these messages and niche content (specific medical publications, research papers, subscription requests to medical journals, etc.) favorably.

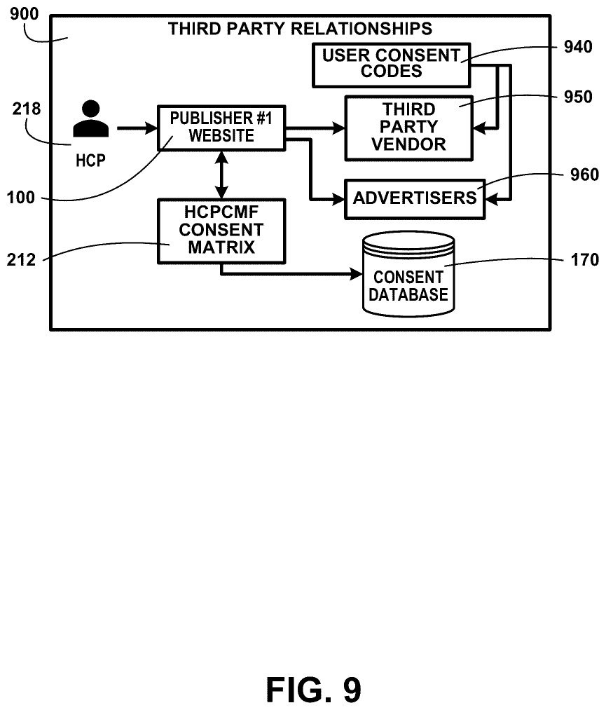

FIG. 9 shows a block diagram of an overview of third-party relationships of one embodiment. FIG. 9 shows building trust-based third-party relationships 900 with the HCP 218 and publisher #1 website 100. The HCPCMF [symbol] consent matrix 212 user consent codes 940 generate consent forms for the third-party vendor 950 and advertisers 960 to seek consent from their users to maintain regulatory compliance.

Whenever HCPs provide their consent, these consented parameters, within the HCPCMF are automatically generated into what is known as ‘User Consent Codes’ that the respective Publishers can share with third-party entities, further building the trust/relationship between the concerned Publisher with their respective third-party relations, that is, vendors. Thus, HCPCMF enables Publishers within the Ad Tech space to expand their scope of third-party relations, based on transparency, and compliance, and thereby augment their trustworthiness in the business domain/industry. The HCPCMF [symbol] consent matrix 212 also stores the HCP 218 consent form responses on the consent [symbol] database 170 of one embodiment.

To meet specific criteria, publishers frequently need to collaborate with third-party suppliers and vendors. The HCP Consent Management Framework 200 of FIG. 2 also supports ensuring that HCP 218 consent is applied to these third parties, as well as that of those business organizations that adhere to the specified privacy and data protection rules. For example, if an HCP 218 withdraws its consent, the consequences are applied to any third party with whom the publisher 210 of FIG. 2 shared HCP 218 data. Furthermore, processing of HCP 218 data ceases immediately in such a circumstance, and neither the publisher 210 of FIG. 2 nor any of their third-party vendors or data processors can thereafter process that HCP's data.

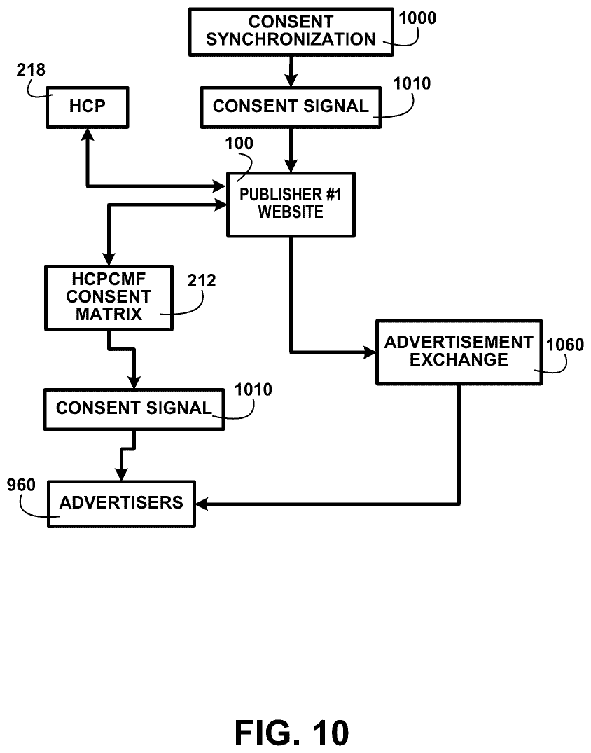

FIG. 10 shows for illustrative purposes only an example of consent synchronization of one embodiment. FIG. 10 shows consent synchronization 1000 using a consent signal 1010 when an HCP 218 user accesses the publisher #1 website 100. The HCPCMF consent matrix 212 sends through the wireless communications the consent signal 1010 to advertisers 960 and an advertisement exchange 1060 to determine if advertising medical product content information is within compliance and covered by HCP 218 user consent of one embodiment.

The HCP Consent Management Framework 200 of FIG. 2 supports the synchronization of permission signals between publishers, advertisers, and advertising exchanges to assure compliance. This is also the more crucial advertisement delivery in a programmatic advertising ecosystem where advertising impressions and placement of these advertisements take place in the blink of an eye.

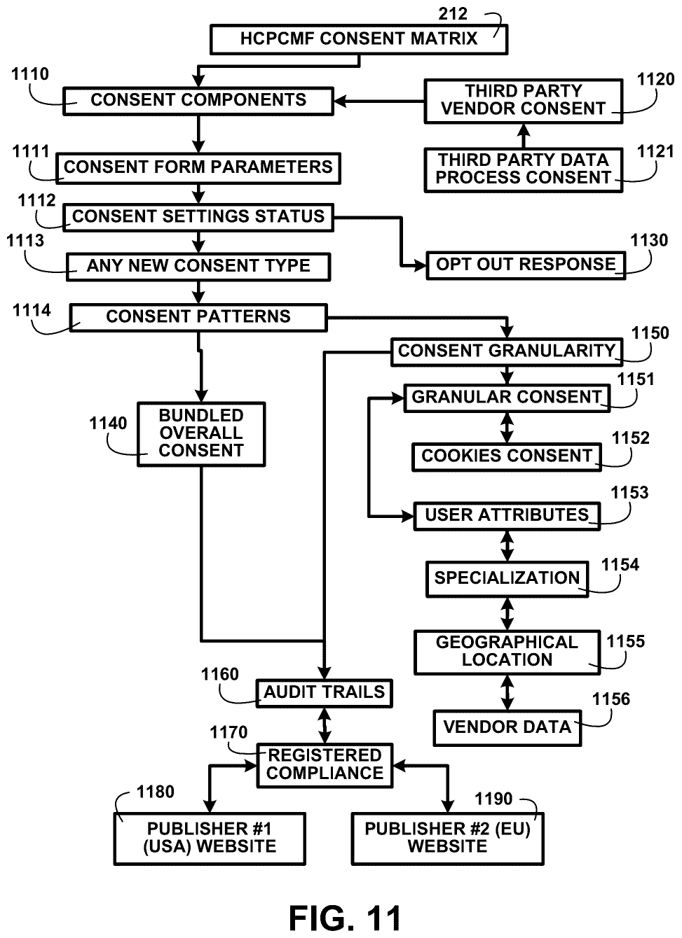

FIG. 11 shows a block diagram of an overview of the consent matrix of one embodiment. FIG. 11 shows the HCPCMF consent matrix 212 consent components 1110. The consent components 1110 include third-party vendor consent 1120 selection for third-party data process consent 1121. The consent components 1110 include consent form parameters 1111 and consent settings status 1112.

The consent settings status 1112 may show an HCP 218 of FIG. 2 opt-out response 1130. The consent component 1110 includes any new consent type 1113, for example, that was published by a regulatory agency. The consent components 1110 include consent patterns 1114. In one embodiment, a consent pattern 1114 may include a bundled overall consent 1140 wherein the HCP 218 of FIG. 2 user accepts all medical product content information. In another embodiment, consent patterns 1114 provide consent granularity 1150 wherein a granular consent 1151 form may include cookies consent 1152 and specific user attributes 1153.

In one embodiment, user attributes 1153 include specialization 1154, geographical location 1155, and vendor data 1156. The HCPCMF consent matrix 212 maintains consent audit trails 1160 for registered compliance 1170. Different geographical locations have differing compliance regulations. However, the HCPCMF consent matrix 212 maintains consent audit trails 1160, for example, a publisher 1 (USA) website 1180 and a publisher 2 (EU) website 1190 of one embodiment.

The HCP Consent Management Framework 200 of FIG. 2 provides a one-stop destination for publishers where they can manage all of their HCP 218 of FIG. 2 consent-related components and that too from a single consent management repository and dashboard. Publishers can access the framework to review their current consent settings, which they are offering to their respective HCPs, review these consent-related choices that they offer, and make required changes to these choices as per their specific needs.

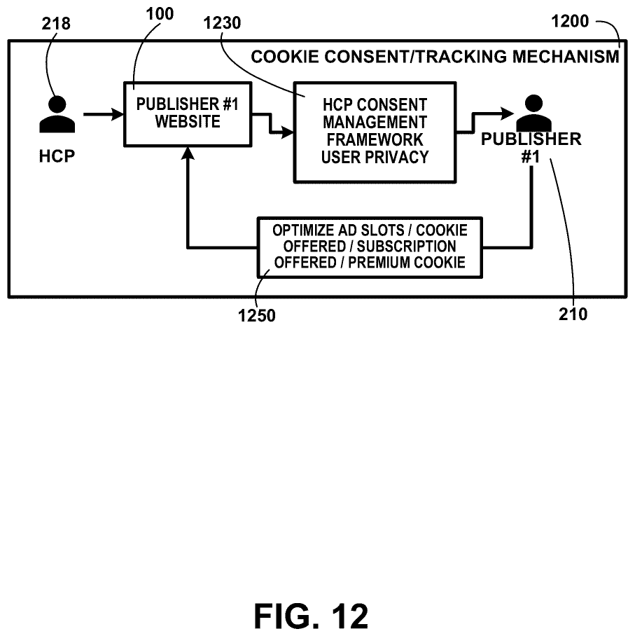

FIG. 12 shows for illustrative purposes only an example of the cookie consent/tracking mechanism of one embodiment. FIG. 12 shows a cookie consent/tracking mechanism 1200. The cookie consent/tracking mechanism 1200 maintains vigilant HCP 218 privacy on a publisher #1 website 100. HCP Consent Management Framework user privacy 1230 is a main concern of the publisher 210. The HCP Consent Management Framework user privacy 1230 uses the platform to optimize ad slots/cookie offered/subscription offered/premium cookie 1250 of one embodiment.

Cookie Consent Management: If cookies or similar tracking mechanisms are used for profiling end-users and sending messages and advertisements based on collected information, publishers need to obtain consent from HCPs and transparently explain the tracking purposes to them.

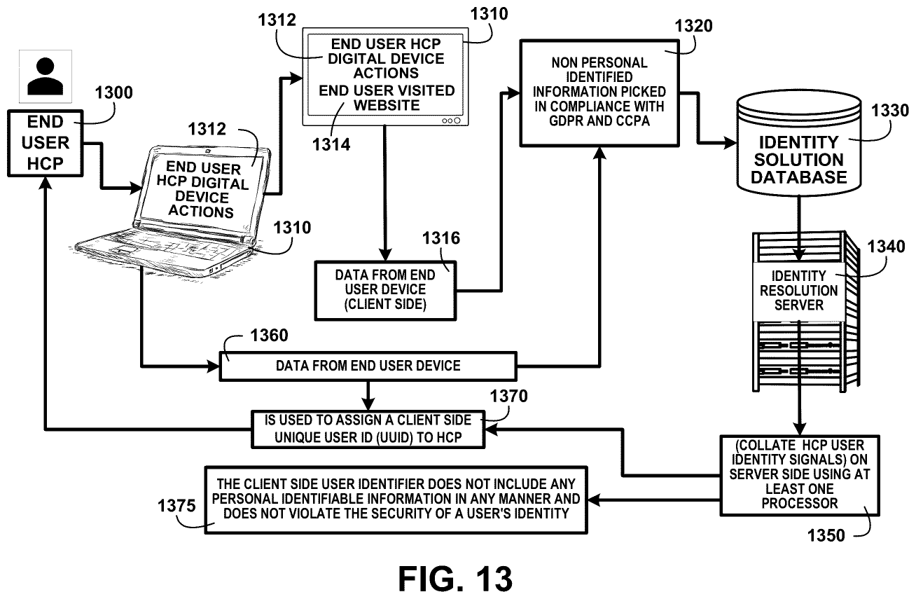

FIG. 13 shows for illustrative purposes only an example of a unique HCP user identity of one embodiment. FIG. 13 shows an end user HCP 1300 having an end user HCP digital device 1310. The end user HCP 1300 performs end-user HCP digital device actions 1312 using the end user HCP digital device 1310 to, for example, an end-user visited website 1314 for data and information searches for data from the end-user device (client side) 1316. Collecting the end user HCP 1300 information includes non-personal identified information picked in compliance with GDPR and CCPA 1320, wherein PIIs refers to Personal Identifiable Information. Wherein GDPR refers to the General Data Protection Regulation and CCPA refers to the California Consumer Privacy Act. The collected end-user HCP 1300 information is stored on an identity solution database 1330. The collected end-user HCP 1300 information is processed on an identity resolution server 1340 to collate HCP user identity signals on the server side using at least one processor 1350 using multiple session and browser attributes such as browser details, page reference, and timestamp, combined with generated random tokens and hashing algorithm to create a non-conflicting client-side unique user identity.

The client-side user identifier does not include any Personal Identifiable Information (PII) in any manner and does not violate the security of a user's identity 1375. Data from end-user device 1360 is used to assign a client-side unique user ID (UUID) to HCP 1370. The unique HCP user identity system solution has reviewed different facets of the HCP identity resolution jigsaw and has produced an innovative solution in the form of a client-side (anything that happens at the end user's device is commonly referred to as client side) unique identifier within the user's browser or the application that these HCPs are using.

Furthermore, the unique HCP user identity system solution has successfully developed a Server-side user (HCP) profile unification mechanism that utilizes various identity signals such as logged-in user information, which includes additional data points such as email ID, mobile ID, HCP ID, universal ID, device ID, etc. of one embodiment.

Specific websites the HCP access to garner knowledge or they visit specific blogs or paid content to access particular articles and case studies, to upgrade their awareness around specific medical topics. HCP subscriptions to distinct online journals and scientific publications; associations with certain medical associations, institutions, and libraries. The type of articles the HCP prefers reading. Constantly reviewing new drugs and formulations that are being developed by pharma companies for specific ailments. The HCP is specifically intrigued about certain human conditions and is seeking the latest drugs that have just entered the market or are in the stage of being developed for treating these disorders.

These HCP professionals are actively looking to stay abreast with the latest developments that are taking place in the medical, pharmaceutical, and life sciences domains. These HCPs want to go forward and undergo new and advanced certifications, training, or learnings, for specific health conditions of patients that whom these HCPs provide treatments. All medicines, drugs, and formulations they prefer over a range of drugs belonging to a particular drug category, and so on.

The aforementioned are just but a fraction of the distinct behavioral footprints that HCPs are leaving behind in the digital firmament during their daily interaction with online touch points be it their personal devices, the apps on these devices, the healthcare systems they use, the websites they visit, the EHRs and POCs they are associated with, etc.

The exponential wealth of data that is thus generated by the HCP community is a veritable treasure trove that can be put to good use. This data has beneficial use especially for augmenting existing pharma messaging opportunities, exploring, and constructing data for enriching HCP personas, and developing specific segmented targeted audiences that can be employed by pharma marketers to bolster the effectiveness of their advertising campaigns.

Though the potential and possibilities afforded by HCP-related data are abundant there is a major roadblock that prevents effective utilization of this vast wealth of information for commercial repurposing. To begin with, the need to identify various digital touch points of an HCP and then using those digital touchpoints to confirm their identity in the affirmative. Once that part is taken care of go ahead and discover their specific behavioral patterns during their caregiving journeys that occur daily.

Identity resolution would also entail the synchronization of an HCP's profile that is offline as well as online. Once the identity of an HCP is established definitively then their persona or profile can be built that would then be improved with time with more behavioral patterns being added to the persona/profile and utilized accordingly for commercial endeavors.

Another facet of identity resolution is to generate and then assign a unique user identity (UUID) to the identified HCPs and then use this UUID as an umbrella for that particular HCP within which the individuals' behavioral patterns are collated and enhanced on the go and utilized for business use within healthcare-related advertising technology. Moreover, various digital footprints of an HCP need to be identified, and then these identifiers ought to be brought together and provided a UUID for all future referencing and to prevent identity-related conflicts from arising. Upon assigning the UUID for an HCP, the HCP unique UUID is then added to the existing HCP consent forms and future consent forms for the newly determined advertising targeting.

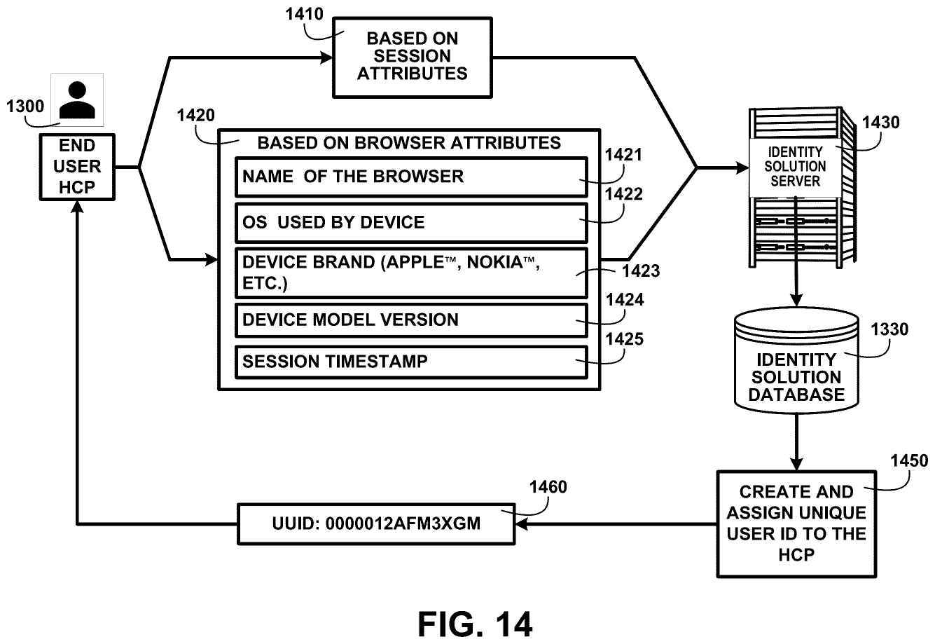

FIG. 14 shows for illustrative purposes only an example of client-side user identifiers of one embodiment. FIG. 14 shows the end-user HCP 1300 information collected data based on session attributes 1410 including based on browser attributes 1420 for example, name of the browser 1421, operating system OS used by device 1422, device brand (Apple™, Nokia™, etc.) 1423, device model version 1424, and session timestamp 1425. The data is transmitted to an identity solution server 1430 for processing and stored in the identity solution database 1330. The processed end user HCP 1300 information collected data is used to create and assign a unique user ID to the HCP 1450, for example, UUID: 0000012afm3xgm 1460 of one embodiment.

The client-side identifier is created using various sessions and browser attributes, which do not include any PIls (Personal Identifiable Information) in any manner. Thus, the solution does not violate the security of a user's identity and thereby stays in compliance with the data security prescriptions of various data security compliance frameworks. Moreover, the client-side identifier that is generated or created by the unique HCP user identity system solution is unique for every end user/HCP.

Multiple session and browser attributes such as browser details, page reference, and timestamp, combined with generated random tokens and hashing algorithm to create a non-conflicting client-side unique user identity.

Other than session attributes, there are various browser-based attributes such as the name of the browser (Google Chrome™, Apple Safari™, Microsoft Edge™ Mozilla Firefox™, etc.) that the HCP is using on their device to browser a particular website, the OS used by the user's device (Windows™, macOS™, Android™, iOS™, etc.), the type of device (laptop, tablet, mobile phone, etc.) that the HCP is currently using, the version of the browser used on the user's device, the brand of the device used (manufacturer such as Apple™, Google™, Samsung™, Nokia™, etc.), the device model (Apple iPhone 14 Pro Max™, Samsung Galaxy A14™, etc.), the version of the device model used by the HCP (Apple iPhone 14™, Apple iPhone 13™, Apple iPhone 12™, etc.), session timestamps (The time span or the session that a particular HCP spend on a particular website for doing different activities or actions thereof. A particular online session can last anywhere between a few seconds, to a few minutes, or even for an hour or more.), are all identified and then duly picked up and utilized accordingly for generating a client-side unique user identifier, which is the Unique User Identity (UUID) of one embodiment.

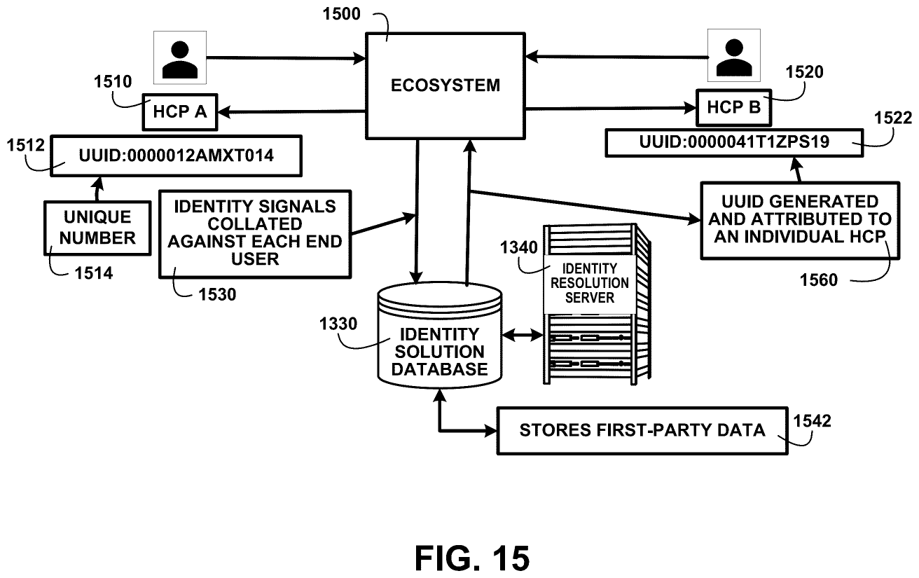

FIG. 15 shows for illustrative purposes only an example of one HCP user and one UUID of one embodiment. FIG. 15 shows an ecosystem 1500. The unique user ID assigned, for example, to two HCPs, is shown for HCP A 1510 UUID:0000012amxt014 1512 and a different unique number 1514 than HCP B 1520 UUID:0000041t1zps19 1522. The unique user ID is applied to identity signals collated against each end user 1530 and stored in the identity solution database 1330. The identity solution database 1330 stores first-party data 1542 that is processed in the identity resolution server 1340. The UUID generated and attributed to an individual HCP 1560 of one embodiment.

Once generated the UUID is tied or mapped with a specific end user, which means that every HCP associated with the ecosystem has a unique identity. As per this 1:1 (one-is-to-one) arrangement, a particular HCP within the ambit of the ecosystem will never have two different identities whatsoever. Also, additional attributes of the user are associated with their specific UUID in the future, thereby further enriching the user's current identity attributes within the ecosystem. The enrichment of a user's identity is a dynamic and ongoing process and will continue up until the ecosystem persists.

This unique user identifier, that is, UUID to begin with is saved as first-party data, which provides us with a natural browser-side persistence phenomenon. This identifier is then ported to the Identity Resolution server and all other attributes collected associated with a particular HCP user are linked to this identifier accordingly. Moreover, the UUID becomes a proprietary identification to map the user's online digital footprint signals and even offline user-related data, henceforth.

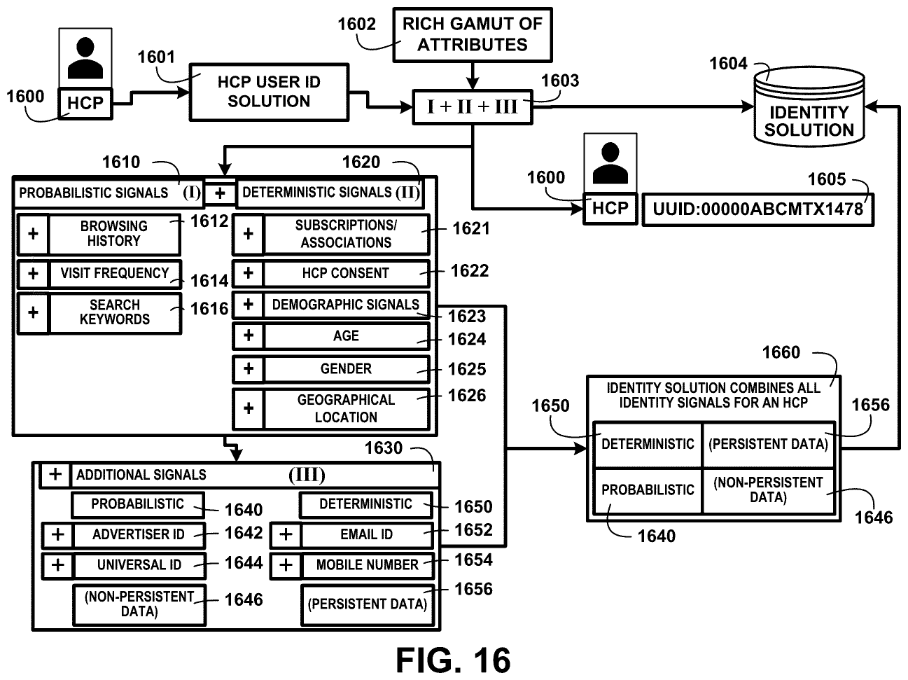

FIG. 16 shows for illustrative purposes only an example of probabilistic and deterministic identifiable signals of one embodiment. FIG. 16 shows an HCP 1600 data collection using the HCP user ID solution 1601. The data collection is highly detailed using a rich gamut of attributes 1602 as described, for example, in I+II+III 1603 of the identity solution 1604 for the UUID:00000abcmtx1478 1605 of HCP 1600. The attributes include HCP 1600 probabilistic signals (I) 1610 including browsing history 1612, visit frequency 1614, and search keywords 1616. Attributes include HCP 1600 deterministic signals (II) 1620 including subscriptions/associations 1621, HCP consent 1622, demographic signals 1623, age 1624, gender 1625, and geographical location 1626.

The rich gamut of attributes 1602 also includes additional signals (III) 1630. The additional signals (III) 1630 include probabilistic 1640 signals of advertiser ID 1642, and universal ID 1644, representing non-persistent data 1646. The additional signals (III) 1630 include deterministic 1650 signals of email ID 1652, and mobile number 1654, which represents persistent data 1656. The identity solution combines all identity signals for an HCP 1660, for example, deterministic 1650, (persistent data) 1656, probabilistic 1640, and (non-persistent data) 1646 of one embodiment.

The UUID is built out based on two primary types of user identity signals, namely, deterministic identifiable signals and probabilistic identifiable signals associated with a particular end-user or HCP. Here is a description of these two distinct types of user identity signals that UUID combines together to create a unique user identity for HCPs.

There are several deterministic identifiable signals associated with an end user that help determine that the end user is indeed a medical practitioner or an HCP. Here are some of the deterministic identifiable signals of online users that help establish the fact that they are indeed HCPs. The actions or activities conducted by end users on a website provide ample evidence that they are part of the medical fraternity. This would include taking subscriptions for online or offline medical journals or seeking association with various medical institutions, bodies, or libraries/digital content.

Probabilistic identifiable signals associated with an end-user include probable indicators or signals about an end user, which point out that the said user is indeed an HCP, or a person associated with the medical fraternity in one way or another. These are some of the probabilistic identifiable signals of an online user that indicate that the user is an HCP: the browsing history of the end user provides a vital clue about their profession; the frequency of visiting websites associated with and displaying life sciences, pharmaceutical, and medical specific content also suggests and gives away the professional identity of the end user; moreover observing the browsing history of the end user in conjunction with the frequency of their visits to specific websites and use of search keywords, put together provides a positive inkling towards the specialization that an HCP is pursuing as their preferred career path or professional expertise.

Moreover, the unique HCP user identity system solution utilizes numerous Machine Learning algorithms to derive a professional identity with a defined level of confidence based on the probability of identifiable signals that are received.

Various online transactions such as taking admission to specialized medical-specific online courses, course content, etc., also denote the fact that the end user is in some manner associated with the medical or life sciences domains. The explicit consent-based information/details provided by end users for accessing specific content on different websites confirms the fact that the end user is positively an HCP by profession. For example, the HCP is actively seeking medical or life sciences-related information from the websites where they have provided explicit consent. The demographic information of the end user such as their age, gender, and geographical location (specifically the location of their place of work such as a pathological laboratory, a healthcare facility, a private healthcare center, a hospital, a hospice, a sanatorium, any healthcare facility, etc.), all represent positive deterministic characteristic about the end user's profession that they are an HCP.

The UUID for any HCP user is created considering and combining both probabilistic as well as deterministic signals. Furthermore, additional identifiable signals be they probabilistic or deterministic in nature, such as the email ID of the end user and their mobile number (both of which are deterministic indicators that are persistent over a great length of time/over the years) as well as signals such as advertiser IDs, and universal IDs (both of which being probabilistic user identifiers but non-persistent data which is temporary or evanescent in nature), gets associated with a UUID over a given period of time. This enables the unique HCP user identity system solution to define a rich collection of user-attributable data points or data dimensions against a user's identity and utilize the enriched user identities to augment a host of business processes.

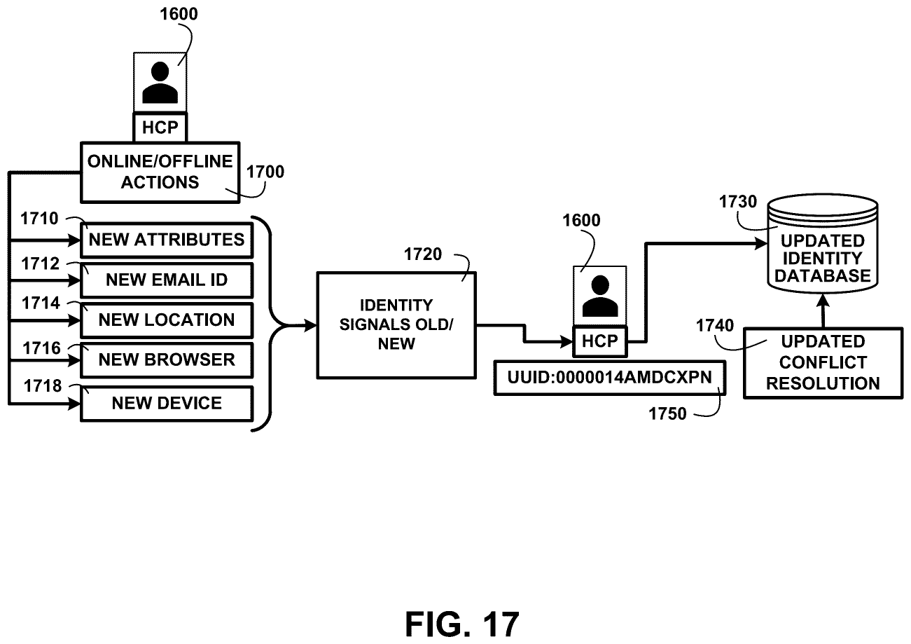

FIG. 17 shows for illustrative purposes only an example of identity enrichment of one embodiment. FIG. 17 shows the HCP 1600 online/offline actions 1700. The updating of HCP attributes includes the online/offline actions 1700 which, for example, includes new attributes 1710, new email ID 1712, new location 1714, new browser 1716, and new device 1718. Identity signals old/new 1720 of the HCP 1600 are used to maintain an updated identity database 1730, and updated conflict resolution 1740 for the HCP 1600 identified by the UUID:0000014amdcxpn 1750 of one embodiment.

There could be HCP's activity corresponding to these persistent and non-persistent identifiers from other online and offline actions which can therefore be attributed to UUID once it is created. Over a span of time when additional attributes are associated with the user's UUID, some of these attributes such as work location, device type, browser version, browser type, email ID, and the like may change. However, the UUID not only incorporates these new attributes but also resolves any conflicts that might arise with new attributes getting added and associated with a user's unique identity over a given period of time.

The creation of a UUID for HCP end users is a bespoke technical innovation that has been ideated and deployed by the unique HCP user identity system solution in the realm of advertising technology and provides us with numerous business-related advantages.

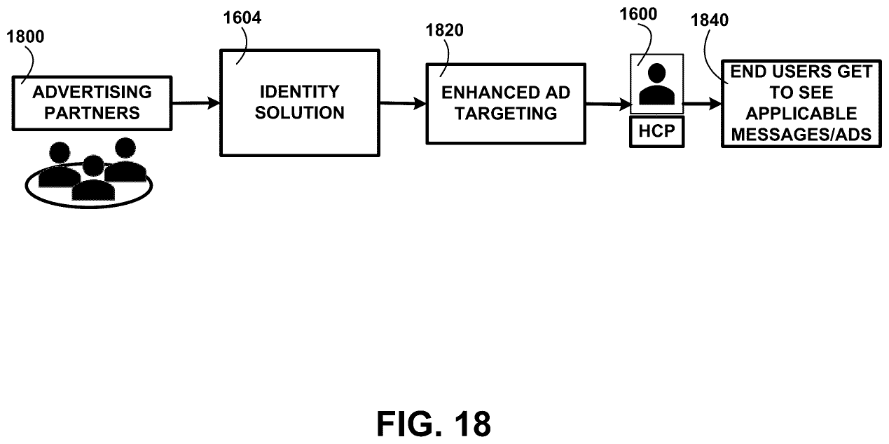

FIG. 18 shows for illustrative purposes only an example of advertising targeting of one embodiment. FIG. 18 shows advertising partners 1800 who benefit from the identity solution 1604. The detailed HCP unique user ID allows enhanced advertising targeting 1820 by the advertising partners 1800 to provide the HCP 1600 specific information they have consented to receive, and additional content specifically targeted to the HCP 1600 profession field of practice and interest. The enhanced advertising targeting allows end users to see applicable messages/ads 1840 saving them valuable time rather than spending time with searches. It further allows the advertising partners 1800 to learn specific areas of interest of the HCP 1600 rather than wasting time and resources sending content that the HCP 1600 has no relevant need or application to the medical conditions treated by the HCP 1600 of one embodiment.

UUID enables us to better understand the behavior of HCP users, their likes and dislikes, their preferences, and interests across different devices and digital platforms. This helps to achieve improved advertising serving and fulfills the requirement of campaigns where specific audiences need to be targeted. These specialized campaigns can be built based on the knowledge of such user identities and their specific attributes. An improvement in advertising targeting results in a corresponding increase in HCP engagement and conversions. This improves the return on advertising spend (ROAS) for the advertising partners.

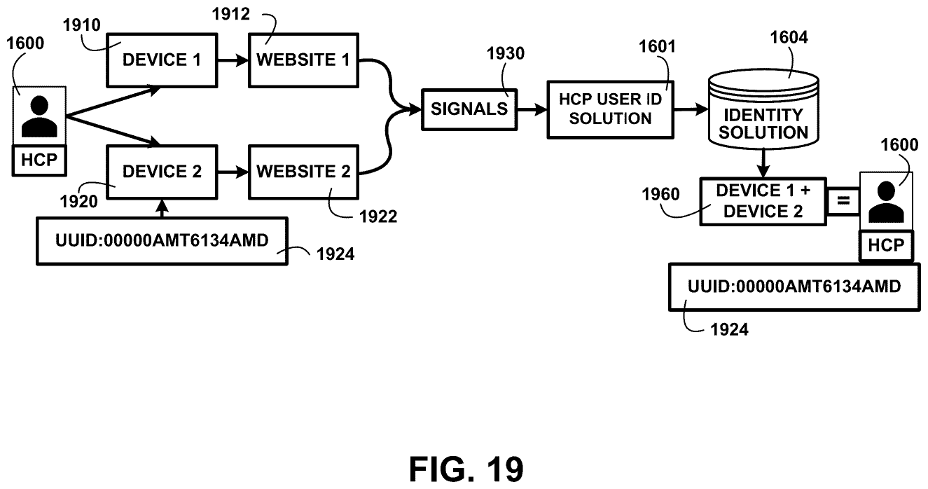

FIG. 19 shows for illustrative purposes only an example of cross-device tracking of one embodiment. FIG. 19 shows the HCP 1600 using multiple devices during the workday in searching for relevant data and information. Device 1 1910 is used by the HCP 1600 to search website 1 1912. For example, later in the workday, the HCP 1600 is using device 2 1920 to search website 2 1922 for a pharma product for treating a particular patient's condition. The HCP 1600 identified by UUID:00000amt6134amd 1924 is transmitting signals 1930 to the HCP user ID solution 1601. The identity solution 1604 processes the device 1+device 2 1960=HCP 1600 UUID:00000amt6134amd 1924 providing the advertising partners 1800 of FIG. 18 with current up-to-the-minute interest by the HCP 1600 to allow them to send specific data content and information for specifically what the HCP 1600 had searched for and provide the HCP 1600 with a product the HCP 1600 may prefer to use to treat the patient's condition of one embodiment.

As user interactions and conversions can be tracked across different user devices, be it smartphones, tablets, laptops, and desktops, this enables the creation and implementation of retargeting and personalized advertising experiences across these devices.

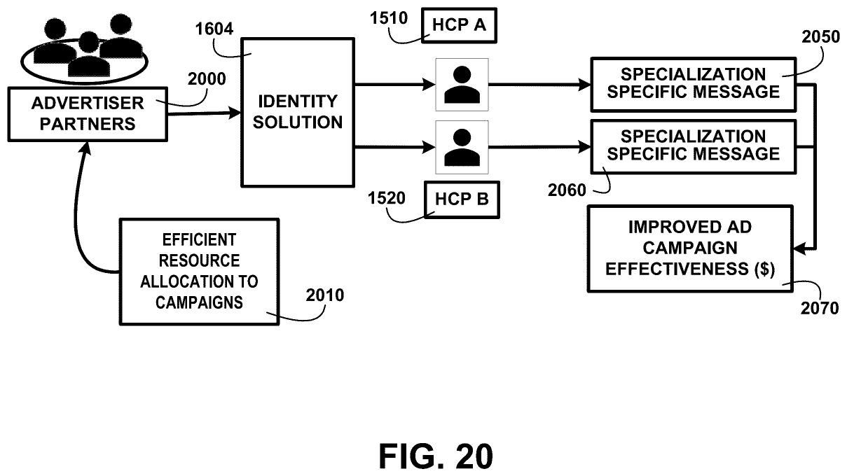

FIG. 20 shows for illustrative purposes only an example of personalization (user experience) of one embodiment. FIG. 20 shows advertiser partners 2000 efficient resource allocation to campaigns 2010 using the identity solution 1604. The identity solution 1604 provides the advertiser partners 2000 the capability to personalize content, for example, HCP A 1510 and HCP B 1520. The specialization-specific message 2050 to HCP A 1510 can include advertising content specifically for the data and information being sought by HCP A 1510 to enhance the HCP A 1510 user experience with the advertiser partner.

Likewise, the specialization-specific message 2060 to HCP B 1520 may be in a completely different specialization as identified by the identity solution 1604 and the advertiser partner with current and newly developed available products can send the advertising content and data to HCP B 1520. The improved advertising campaign effectiveness ($) 2070 both in specifically targeted advertisements and timely delivery of product data and information that builds solid relationships with the healthcare professionals who will appreciate the prompt response to meet their needs of one embodiment. Equipped with a rich gamut of user attributes, the identity solution enables the personalization of advertising experiences. HCPs are more likely to engage in advertising that is relevant to their interests and needs, resulting in a better user experience.

Our advertising partners can boost the overall effectiveness of their advertising campaigns based on user insights derived from the audience targeting lists based on the UUID. This results in augmentation of advertising campaign performance, higher engagement, as well as an increase in the user conversion rates and improvement of brand loyalty.

Utilizing information around UUID HCPs partners can allocate advertising inventory and budgets more efficiently by targeting specific user segments with a higher likelihood of conversion. This reduces advertising spending on less relevant audiences and wastage of inventory and the corresponding advertising expenditure.

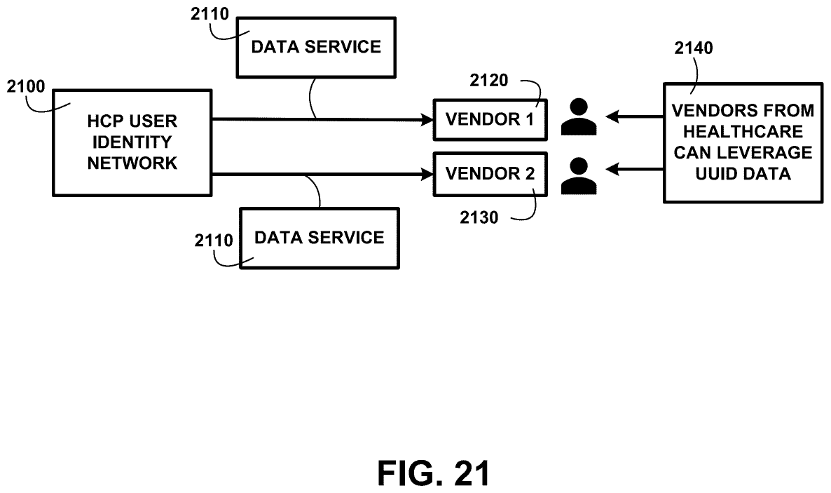

FIG. 21 shows for illustrative purposes only an example of data monetization of one embodiment. FIG. 21 shows an HCP user identity network 2100 that provides data service 2110 of the identity solution 1604 of FIG. 16 to multiple advertiser partners 2000 of FIG. 20 simultaneously. Data service 2110 to vendor 1 2120 and at the same time data service 2110 to vendor 2 2130 means the vendors from healthcare can leverage UUID data 2140 to utilize UUID data to generate additional revenue streams for the unique HCP user identity system by offering data monetization services to other businesses looking for user insights that are specific to the healthcare and life sciences domains of one embodiment.

The robust privacy controls built into the UUID generation journey helps the unique HCP user identity system to always stay in compliance with data protection regulation agencies such as GDPR (General Data Protection Regulation), CCPA (California Consumer Privacy Act), etc., by ensuring HCP consent and data handling practices are in line with the prescribed legal requirements of the unique HCP user identity system market is operating in, across the globe.

This particular solution provides a significant competitive advantage in the advertising technology domain. It allows the unique HCP user identity system to offer more sophisticated targeting and analytics capabilities to the business partners be it advertisers or publishers. By providing personalized and relevant messages to end users utilizing UUID capabilities helps the unique HCP user identity system to build stronger, long-term relationships with the advertiser and publisher partners. The information that is collected and maintained in the Identity Server provides valuable insights into HCP user behavior, helping the unique HCP user identity system business partners to define their existing advertising strategies and product offerings.

On the Publisher side, the unique HCP user identity system can cater to their specific requirement for allowing or restricting certain audiences to specific content. This can easily be enabled using the identity solution. The restriction of certain segments of the audience is also dependent on the quality of the audience which in turn is based on the confidence level of the unique HCP user identity system identity resolution capabilities.

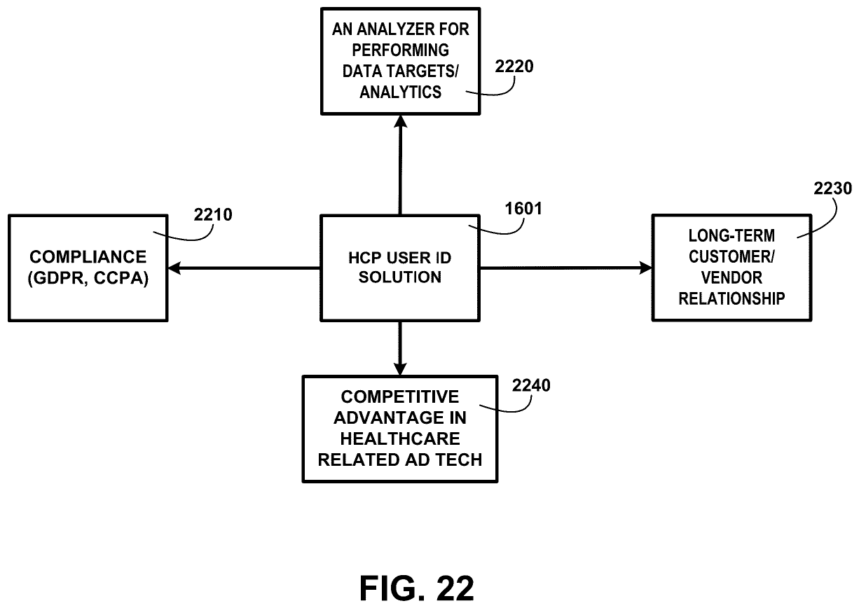

FIG. 22 shows a block diagram of an overview of other benefits of one embodiment. FIG. 22 shows the HCP user ID solution 1601 of FIG. 16 maintaining compliance (GDPR, CCPA) 2210 requirements. An analyzer for performing data targets/analytics 2220 to assist multiple advertiser partners 2000 of FIG. 20. The identity solution 1604 of FIG. 16 contributes to building long-term customer/vendor relationships 2230. The identity solution 1604 of FIG. 16 provides a competitive advantage in healthcare-related advertising technology 2240 that benefits multiple advertiser partners 2000 of FIG. 20 of one embodiment.

The UUID also persists in third-party data, which enables a client-side sync between the first and third-party data attributes for an end user (HCP). This syncing of data facilitates the persistence and scaling of the user's identity across multiple domains. This also results in the server side collecting a wider footprint of user signals against the same user identity enriching the existing user identity attributes even further.

The foregoing has described the principles, embodiments, and modes of operation of the present invention. However, the invention should not be construed as being limited to the particular embodiments discussed. The above-described embodiments should be regarded as illustrative rather than restrictive, and it should be appreciated that variations may be made in those embodiments by workers skilled in the art without departing from the scope of the present invention as defined by the following claims.

What is claimed is:

1. A unique healthcare professional user identity system, comprising:
an identity solution database configured for collecting end-user healthcare professional (HCP) digital device action signals;
an identity resolution server coupled to the identity solution database configured to collate the client-side end-user HCP digital device action signals;
at least one processor coupled to the identity resolution server configured for performing data targets/analytics of the end user HCP digital device actions signals;
wherein the at least one processor coupled to the identity resolution server is further configured to identify behavioral patterns based on browser attributes of the end user HCP digital device actions signals;
wherein the at least one processor is further configured to generate and assign a unique user identity (UUID) to the identified HCP, the HCP-identified behavioral patterns, and existing HCP consent forms and future consent forms; and
wherein the identity resolution server is further configured to provide the behavioral patterns data to advertiser partners for the newly determined advertising targeting-related data.

2. The unique healthcare professional user identity system of claim 1, further comprising identified HCP browser attributes deterministic signals include subscriptions/associations, HCP consent, demographic signals, age, gender, and geographical location.

3. The unique healthcare professional user identity system of claim 1, further comprising identified HCP browser attributes probabilistic signals including browsing history, visit frequency, and search keywords.

4. The unique healthcare professional user identity system of claim 1, further comprising the end user HCP collected data includes browser attributes including the name of the browser, operating system OS used by the device, device brand, device model version, and session timestamp.

5. The unique healthcare professional user identity system of claim 1, further comprising the identity solution combines all identity signals for an HCP including deterministic persistent data and probabilistic non-persistent data.

6. The unique healthcare professional user identity system of claim 1, further comprising the end user HCP collected data includes additional signals including probabilistic signals of advertiser ID, universal ID, and non-persistent data.

7. The unique healthcare professional user identity system of claim 1, further comprising the end user HCP collected data includes additional signals including deterministic signals of email ID, mobile number, and persistent data.

8. The unique healthcare professional user identity system of claim 1, further comprising HCP attributes updates including online/offline actions that includes new attributes, new email ID, new location, new browser, and new device.

9. The unique healthcare professional user identity system of claim 1, further comprising HCP attributes updates of identity signals old/new of the HCP is used to maintain an updated identity database, and updated conflict resolution for the HCP identified by the UUID.

10. A unique healthcare professional user identity system, comprising:
an identity solution database configured for collecting end-user healthcare professional (HCP) digital device action signals;
an identity resolution server coupled to the identity solution database configured to collate the client-side end-user HCP digital device action signals;
at least one processor coupled to the identity resolution server configured for performing data targets/analytics of the end user HCP digital device actions signals;
wherein the at least one processor coupled to the identity resolution server is further configured to identify behavioral patterns based on browser attributes of the end user HCP digital device actions signals;
wherein the at least one processor is further configured to generate and assign a unique user identity (UUID) to the identified HCP, the HCP-identified behavioral patterns, and existing HCP consent forms and future consent forms;
wherein the identity solution database is further configured for collecting HCP digital device action signals with cross-device tracking of HCP multiple devices used during the workday in searching for relevant data and information; and
wherein the identity resolution server is further configured to provide the behavioral patterns data to advertiser partners for the newly determined advertising targeting-related data.

11. The unique healthcare professional user identity system of claim 10, further comprising identified HCP browser attributes deterministic signals include subscriptions/associations, HCP consent, demographic signals, age, gender, and geographical location.

12. The unique healthcare professional user identity system of claim 10, further comprising identified HCP browser attributes probabilistic signals including browsing history, visit frequency, and search keywords.

13. The unique healthcare professional identity system of claim 10, further comprising the end user HCP collected data includes browser attributes including the name of the browser, operating system OS used by the device, device brand, device model version, and session timestamp.

14. The unique healthcare professional user identity system of claim 10, further comprising the identity solution combines all identity signals for an HCP including deterministic persistent data and probabilistic non-persistent data.

15. A unique healthcare professional user identity system, comprising:
an identity solution database configured for collecting end-user healthcare professional (HCP) digital device action signals;
wherein collecting end-user HCP digital device actions signals of non-personal identified information picked in compliance with regional privacy regulations;
an identity resolution server coupled to the identity solution database configured to collate the client-side end-user HCP digital device actions signals;
at least one processor coupled to the identity resolution server configured for performing data targets/analytics of the end user HCP digital device actions signals;
wherein the at least one processor coupled to the identity resolution server is further configured to identify behavioral patterns based on browser attributes of the end user HCP digital device actions signals;
wherein the at least one processor is further configured to generate and assign a unique user identity (UUID) to the identified HCP, the HCP-identified behavioral patterns, and existing HCP consent forms and future consent forms;
wherein the identity solution database is further configured for collecting HCP digital device action signals with cross-device tracking of HCP multiple devices used during the workday in searching for relevant data and information; and
wherein the identity resolution server is further configured to provide the behavioral patterns data to advertiser partners for the newly determined advertising targeting-related data.

16. The unique healthcare professional identity system of claim 15, further comprising identified HCP browser attributes deterministic signals include subscriptions/associations, HCP consent, demographic signals, age, gender, and geographical location.

17. The unique healthcare professional user identity system of claim 15, further comprising identified HCP browser attributes probabilistic signals including browsing history, visit frequency, and search keywords.

18. The unique healthcare professional user identity system of claim 15, further comprising the end user HCP collected data includes browser attributes including the name of the browser, operating system OS used by the device, device brand, device model version, and session timestamp.

19. The unique healthcare professional user identity system of claim 15, further comprising the identity solution combines all identity signals for an HCP including deterministic persistent data and probabilistic non-persistent data.

20. The unique healthcare professional user identity system of claim 15, further comprising HCP UUID identified data provides a competitive advantage in healthcare-related advertising technology targeting that benefits multiple advertiser partners.
